(19) United States
(12) Patent Application Publication
(10) Pub. No.: US 2008/0267896 A1
(43) Pub. Date: Oct. 30, 2008

(54) Cosmetic Compositions Comprising At Least One Bis-Urea Derivative

(76) Inventor: Charlotte FELTIN, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/967,567

(22) Filed: Dec. 31, 2007

Related U.S. Application Data
(60) Provisional application No. 60/886,516, filed on Jan. 25, 2007.

(30) Foreign Application Priority Data
Dec. 29, 2006 (FR) 06/56059

(51) Int. Cl. A61K 8/42; A61K 31/17; A61Q 5/00; A61K 36/00

(52) U.S. Cl. 424/70.1; 514/597; 424/776

(57) ABSTRACT

The present disclosure relates to cosmetic compositions comprising at least one continuous liquid fatty phase comprising at least one compound of formula (I),

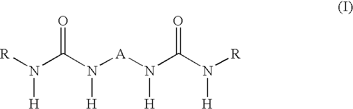

and at least one non-silicone oil with a solubility parameter δₐ ranging from 0 to 5.00 (J/cm³)^{1/2}. The present disclosure also relates to processes for making up or caring for keratin materials, comprising the application to the keratin materials of a composition as described above.

This application claims benefit of U.S. Provisional Application No. 60/886,516, filed Jan. 25, 2007, the contents of which are incorporated herein by reference. This application also claims benefit of priority under 35 U.S.C. § 119 to French Patent Application No. 0656059, filed Dec. 29, 2006, the contents of which are also incorporated herein by reference.

The present disclosure relates to compositions for caring for or making up the skin of either the human face or body, keratin fibers, for instance the eyelashes, the eyebrows, or the hair, or the lips.

Such compositions may be a foundation, a makeup rouge, an eyeshadow, a concealer, a lip cream, a mascara, or a body makeup product, when it is in colored form, or alternatively may be a skincare cream, a hair conditioner, a shampoo, an antisun cream or skin-coloring cream, or a dermatological ointment, when it is in uncolored form.

Care or makeup compositions may have varied textures ranging from fluid to solid. One of the difficulties encountered by users is that of being able to spread the composition uniformly over the entire surface of the face or body so as to distribute the product uniformly. Compositions of thick or solid texture may be difficult to spread on account of their high viscosities. Compositions of fluid texture are not always suitable for obtaining a uniform makeup result, since they do not leave visible marks on the skin, for example on account of their poor spreading over the entire surface of the face to be made up. Creamy textures are thus often sought.

These creamy compositions may contain a thickener that facilitates uptake of the product from the packaging, without loss of product, allowing the product to be distributed homogeneously over the area to be treated or allowing sufficient amounts of product to be taken up, in order to obtain the desired cosmetic effect. When the composition is sufficiently thick, uptake of the product by finger may produce hollows on the surface of the product in the packaging, and, the next time product is taken out, the surface of the product appears as it was at the time of the preceding closure of the jar. In this case, the cream may appear contaminated, giving rise to dissatisfaction on the part of the consumers.

The absence of surface leveling of the cream after each use may occur in particular for a cream of rich and/or thickened texture, which does not flow.

There is thus still a need for compositions for making up, treating, or caring for the skin and keratin fibers, which offer good leveling of the surface in their packaging, giving the impression of opening a new jar on each occasion, and of touching an uncontaminated product.

The inventors have discovered that such compositions can be obtained by combining at least one bis-urea derivative and at least one oil, such as a non-silicone oil.

One embodiment of the present disclosure is directed to cosmetic compositions comprising at least one continuous liquid fatty phase, the continuous liquid fatty phase comprising at least one compound of formula (I):

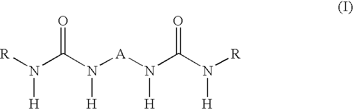

in which:
A is a group of formula:

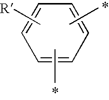

with R′ being a linear or branched C₁ to C₄ alkyl radical and the *s representing the points of attachment of the group A to each of the two nitrogen atoms of the rest of the compound of formula (I), and
R is a saturated or unsaturated, non-cyclic, mono-branched C₆ to C₁₅ alkyl radical whose hydrocarbon-based chain is optionally interrupted with 1 to 3 heteroatoms chosen from O, S and N;
   or a salt or isomer thereof, and at least one non-silicone oil with a solubility parameter δₐ ranging from 0 to 5.00 (J/cm³)^{1/2}.

Another embodiment of the present disclosure is a process for making up or caring for keratin materials, comprising the application to the keratin materials of at least one composition as defined above.

A further embodiment of the present disclosure is a method of using compositions as defined above for obtaining a makeup result that is smooth via leveling of the skin relief and/or matte and/or soft to the touch.

### Hydrocarbon-Based Bis-Ureas

As stated hereinabove, the bis-urea compounds under consideration correspond to the formula (I) below:

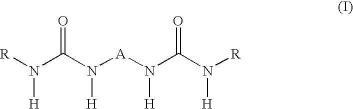

in which:
A is a group of formula:

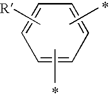

with R′ being a linear or branched C₁ to C₄ alkyl radical and the *s representing the points of attachment of the group A to each of the two nitrogen atoms of the rest of the compound of formula (I), and
R is a saturated or unsaturated, non-cyclic, mono-branched C₆ to C₁₅ alkyl radical whose hydrocarbon-based chain is optionally interrupted with 1 to 3 heteroatoms chosen from O, S and N,
   or a salt or isomer thereof.

According to one embodiment of the disclosure, the group represented by A is a group of formula:

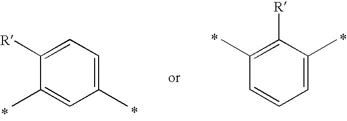

with R′ and the *s being as defined above.

For instance, R′ may be a methyl group, and the group A may then be a group of formula:

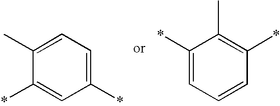

with the *s being as defined above.

According to a first embodiment, R may be chosen from mono-branched radicals of general formula CₙH₂ₙ₊₁, n being an integer ranging from 6 to 15, such as ranging from 7 to 9, for example equal to 8.

Thus, the two groups R of the compound of formula (I) may each represent, independently of each other, a group:

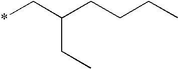

with * representing the point of attachment of each of the groups R to each of the nitrogen atoms of the rest of the compound of formula (I).

According to another embodiment, R may be chosen from mono-branched radicals of general formula Cₘ₋ₚH₂ₘ₊₁₋₂ₚXₚ, p being an integer equal to 1, 2, or 3, for example equal to l, m being an integer ranging from 6 to 15, such as ranging from 10 to 14 or from 10 to 12, for example equal to 11, and X representing sulfur and/or oxygen atoms.

For example, R may be a radical of formula C_{m′}H_{2m′}X—(C_{p′}H_{2p′}X′)ᵣ—CₓH₂ₓ₊₁, in which X and X′ are, independently of each other, an oxygen or sulfur atom, r is 0 or 1, m′, p′, and x are integers such that their sum ranges from 6 to 15, such as from 10 to 12, or for example equal to 11, and at least one of the carbon-based chains C_{m′}H_{2m′}, C_{p′}H_{2p′}, or CₓH₂ₓ₊₁ being branched.

In one embodiment, it is the chain CₓH₂ₓ₊₁ that is branched, r may be equal to 0, m′ may be an integer ranging from 1 to 10, such as from 2 to 6, for example equal to 3, and/or x may be an integer ranging from 4 to 16, such as from 6 to 12, for example equal to 8.

Thus, the two groups R of the compound of formula (I) may each represent, independently of each other, a group:

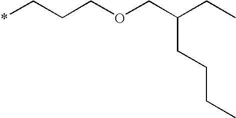

with * representing the point of attachment of each of the groups R to each of the nitrogen atoms of the rest of the compound of formula (I).

Such compounds may be present in the compositions as mixtures with isomers, for example positional isomers on the group A, for example in 95/5 or 80/20 proportions.

As emerges from the embodiments below, the presence of one or the other of these radicals in the molecule of formula (I) may give a universal nature, within the scope of the present disclosure, to the corresponding bis-urea derivatives.

As non-limiting examples of compounds suitable for use in compositions according to the present disclosure, mention may be made of the following compounds, used pure or as a mixture:

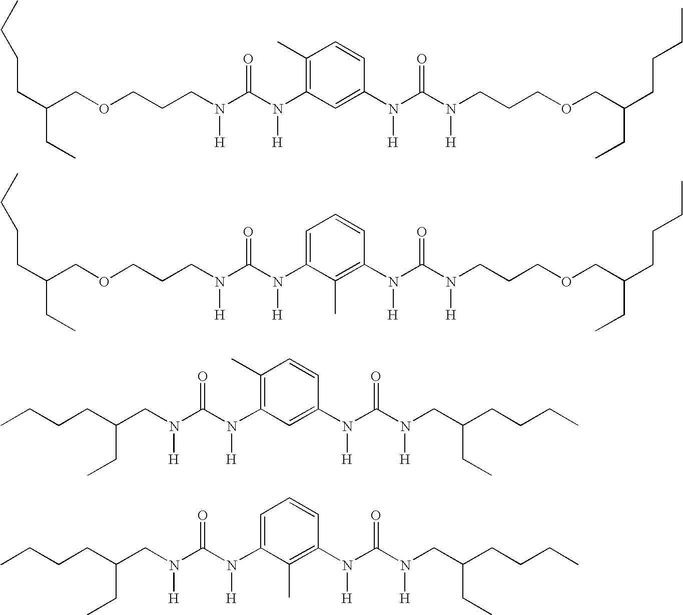

and the salts thereof.

The term “suitable for use in compositions according to the present disclosure” means that the compound of formula (I), either alone or as a mixture in all proportions, may be dissolved in a wide variety of oils and can be effective for texturing the oil or oil mixture and thus for giving it desired physical and/or chemical properties.

The term “physiologically acceptable” denotes a medium free of toxicity, which is compatible with application to the skin, the lips, and/or the integuments of living beings, for example, of human beings. Consequently, compositions according to the present disclosure may be free of compounds that are incompatible with and/or not tolerated for application to the skin, the lips, and/or the integuments.

For the purposes of the present disclosure, the term “effective amount” refers to an amount that is sufficient to obtain texturing of an oil or oil mixture under consideration in compositions according to the present disclosure.

The term “textured liquid fatty phase” denotes a fatty phase in the form of a gel or a thickened liquid. The “textured liquid fatty phase” may flow under its own weight and/or it may be deformed to constant volume if a stress is exerted thereon. This texturing can be reflected by an increase in the viscosity, for example due to the introduction of at least one compound of formula (I).

For example, compositions according to the present disclosure may contain from 0.01% to 20% by weight, such as from 0.1% to 15%, or from 1% to 10%, or from 2% to 8% by weight of compound(s) of formula (I) relative to the total weight of the composition.

The effective amount of compound(s) of formula (I) may represent from 0.01% to 20%, such as from 0.05% to 10%, or from 0.1% to 5%, or from 0.05% to 3%, by weight of the liquid fatty phase.

The effective amount may vary significantly depending, in part, on the nature of the substituent R of the bis-urea derivative, its position, and whether it is used in pure form or as a mixture with other bis-urea derivatives of formula (I), and also on the nature of the liquid fatty phase.

In some embodiments, the compound of formula (I) according to the present disclosure is derived from the reaction between at least one diisocyanate of formula (X):

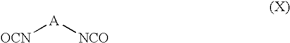

and a primary amine of formula (Y):

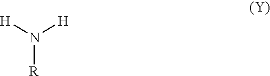

wherein A and R are defined as above.

In some embodiments, the diisocyanates of formula (X) may be positional isomers of the substituent R′ on the group A, for example in 95/5 or 80/20 proportions.

In at least one embodiment, the amine of formula (Y) is used in a mole ratio ranging from 2 to 3 equivalents, such as ranging from 2.1 to 2.5 equivalents, for example 2.2 equivalents per one equivalent of diisocyanate(s) of formula (X). The general reaction scheme is as follows:

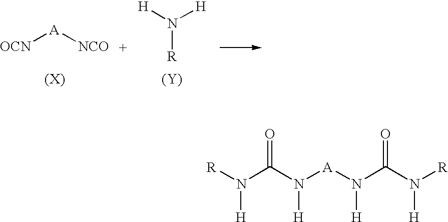

The reaction may be performed under an inert atmosphere, for example under argon, and/or in anhydrous medium with, for example, a reaction medium temperature ranging from 15° C. to 40° C., for example ranging from 18° C. to 25° C.

The diisocyanate(s) of formula (X) may be dissolved in an anhydrous solvent, such as, but not limited to, tetrahydrofuran, 2-methyltetrahydrofuran, N-methylpyrrolidone, butyl acetate, and methyl ethyl ketone, at a concentration which may range from 1% to 30% by weight, such as from 2% to 20%, for example from 4% to 10% by weight.

A solution comprising an amine of formula (Y) can be prepared in the same solvent as the diisocyanate(s) of formula (X) and at a concentration ranging, for example, from 0.1% to 99.9% by mass. In one embodiment of the present disclosure, the temperature of the reaction medium should not exceed 40° C. and the concentration and rate of addition of the solution comprising the amine of formula (Y) is adjusted to these conditions. The reaction medium may be left stirring, for example, for 30 minutes to 12 hours. Monitoring of the reaction progress may be performed by infrared spectrometry (such as by observing the disappearance of the NCO band between 2250 and 2280 cm⁻¹). For example, at the end of the reaction, the reaction medium may be poured into a large amount of acidified water (for example, adjusted to pH 3-4 with HCl). A precipitate can then be obtained, which is filtered off, washed, for example several times, for example with water, and dried under reduced pressure, such as under vacuum or freeze-dried. The precipitate comprises the compounds of formula (II), and may be characterized by NMR spectrometry (such as ¹H and/or ¹³C) and/or by HPLC and may be used without further modification for the texturing of the oily medium under consideration.

In one embodiment, the bis-urea or bis-urea mixture is soluble in the liquid fatty phase to be textured at a temperature of less than or equal to 50° C., or less than or equal to 30° C., for example at room temperature.

In one embodiment, the compounds of formula (I) described above may be admixed with compounds of formula (II) below:

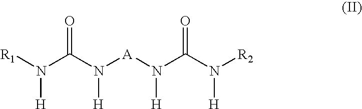

in which:
A is a group of formula:

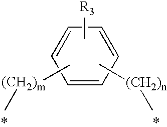

wherein
R₃ is chosen from a hydrogen atom and a linear or branched C₁ to C₄ alkyl radical,
n and m, independently of each other, may be equal to 0 or 1, and
* represents the point of attachment of the group A to the two nitrogen atoms of the residue of the compound of formula (II),
R₁ is chosen from saturated and unsaturated non-cyclic branched C₃ to C₁₅ carbon-based radicals, optionally comprising from 1 to 3 heteroatoms chosen from O, S, F, and N and/or a carbonyl, and combinations thereof,
R₂ is different from R₁ and is chosen from linear, branched and cyclic, saturated and unsaturated C₁-C₂₄ alkyl radicals optionally comprising from 1 to 3 heteroatoms chosen from O, S, F and N, and optionally substituted with:
1, 2, or 3 hydroxyl radicals, and/or
an ester radical (—COOR₄), wherein R₄ is chosen from linear and branched alkyl radicals comprising from 1 to 8, such as from 1 to 6, for example from 2 to 4, carbon atoms; and/or
one or more saturated, unsaturated or aromatic cyclic radicals comprising from 5 to 12 carbon atoms, for example phenyl radicals optionally substituted with one or more groups, which may be identical or different, chosen from C₁-C₄ alkyl and trifluoromethyl radicals, and morpholine derivatives, and/or
one or more linear or branched C₁-C₄ alkyl radicals,
   or a salt or isomer thereof.

In at least one embodiment, n and m may be equal, such as both equal to zero, and R₃ may be a radical R₁₃ as defined below. For example, A may represent a group as shown below:

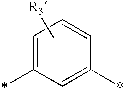

with R₃′ being chosen from linear and branched C₁ to C₄ alkyl radicals and * representing the points of attachment of the group A to the two nitrogen atoms of the residue of the compound of formula (II).

According to one possible embodiment, the compound of formula (II) may comprise, as A, at least one group chosen from:

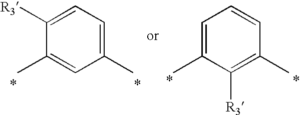

wherein R₃′ and * are defined as above.

In one embodiment, R₃′ may be a methyl group. For example, the group A may represent a group

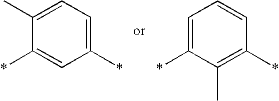

wherein * is as defined above.

In one embodiment of the compounds of formula (II), A is a mixture of 2,4-tolylene and 2,6-tolylene, for example in (2,4 isomer)/(2,6 isomer) proportions ranging from 95/5 to 80/20.

According to one embodiment of the present disclosure, the compound of formula (II) may comprise, as R₁, a branched C₆-C₁₅ radical.

According to one embodiment of the present disclosure, the compound of formula (II) may comprise, as R₁, a group chosen from:

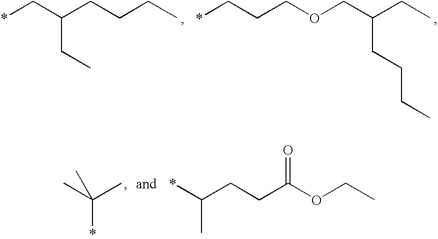

wherein * represents the point of attachment of the group R₁ to the nitrogen of the residue of the compound of formula (II).

As emerges from the non-limiting examples below, the presence of one and/or another of the two radicals in the molecule of formula (II) may give a universal nature within the scope of the present disclosure to the corresponding asymmetric bis-urea derivatives.

R₂, which may be different from R₁, may be chosen from the following groups:

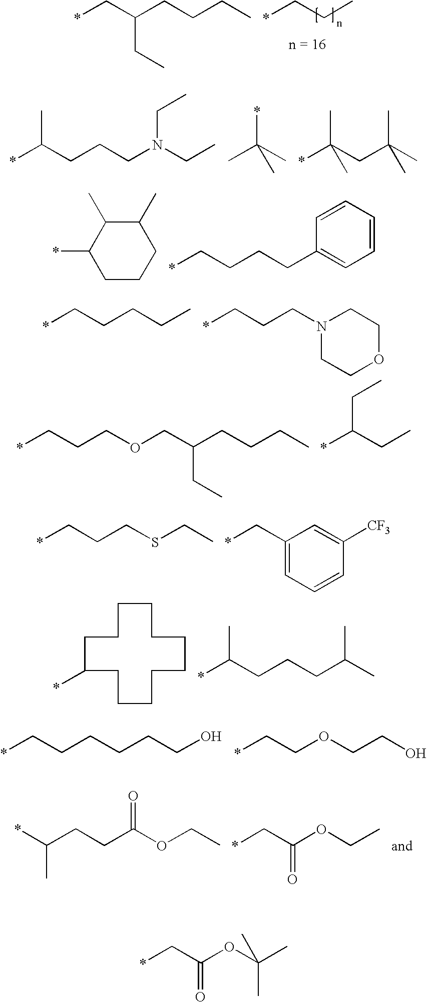

wherein * represents the point of attachment of the group R₂ to the nitrogen of the residue of the compound of formula (II).

Regarding compounds that may be used in compositions according to the present disclosure, mention may be made, without limitation, of the following compounds:

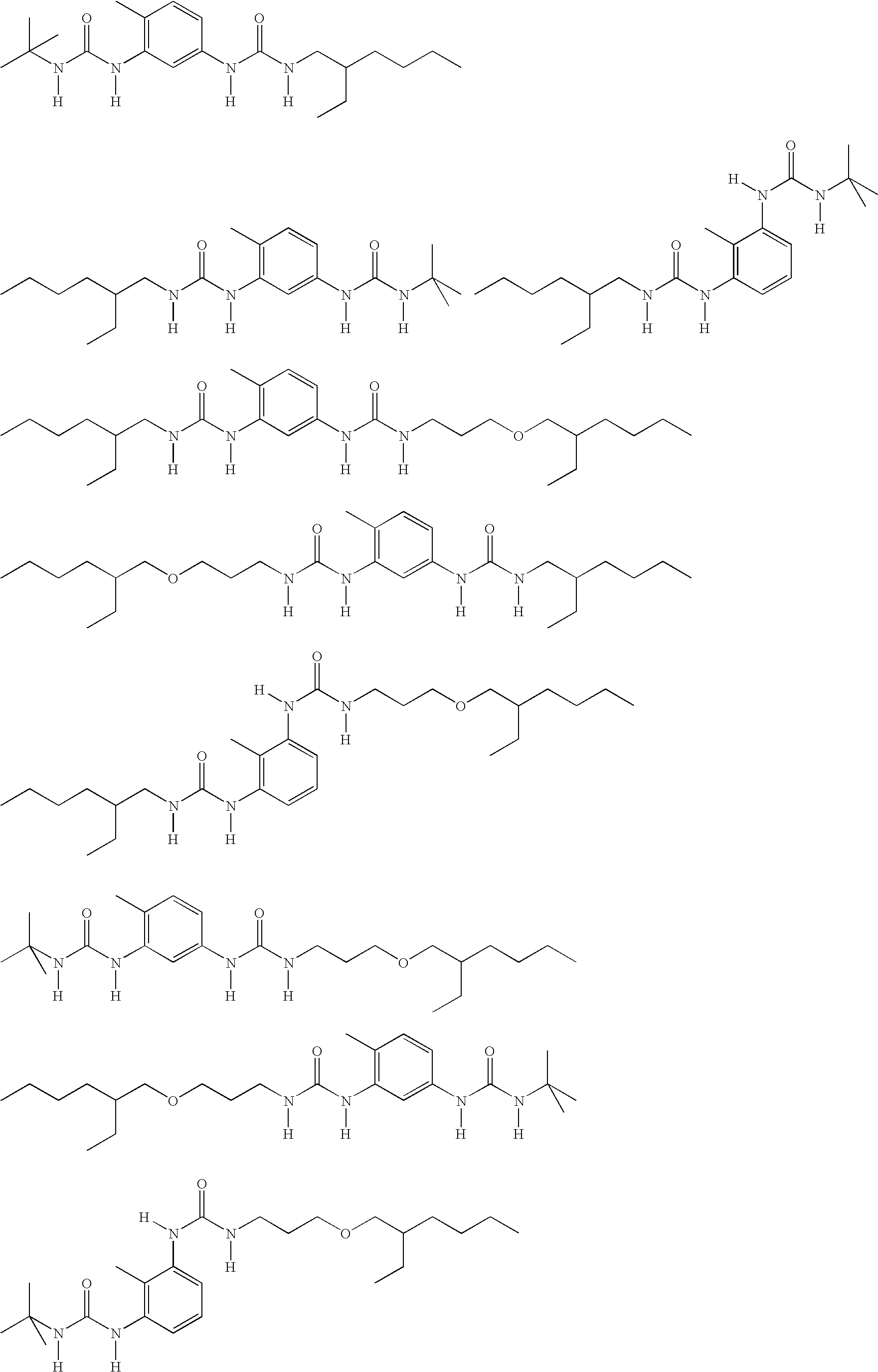

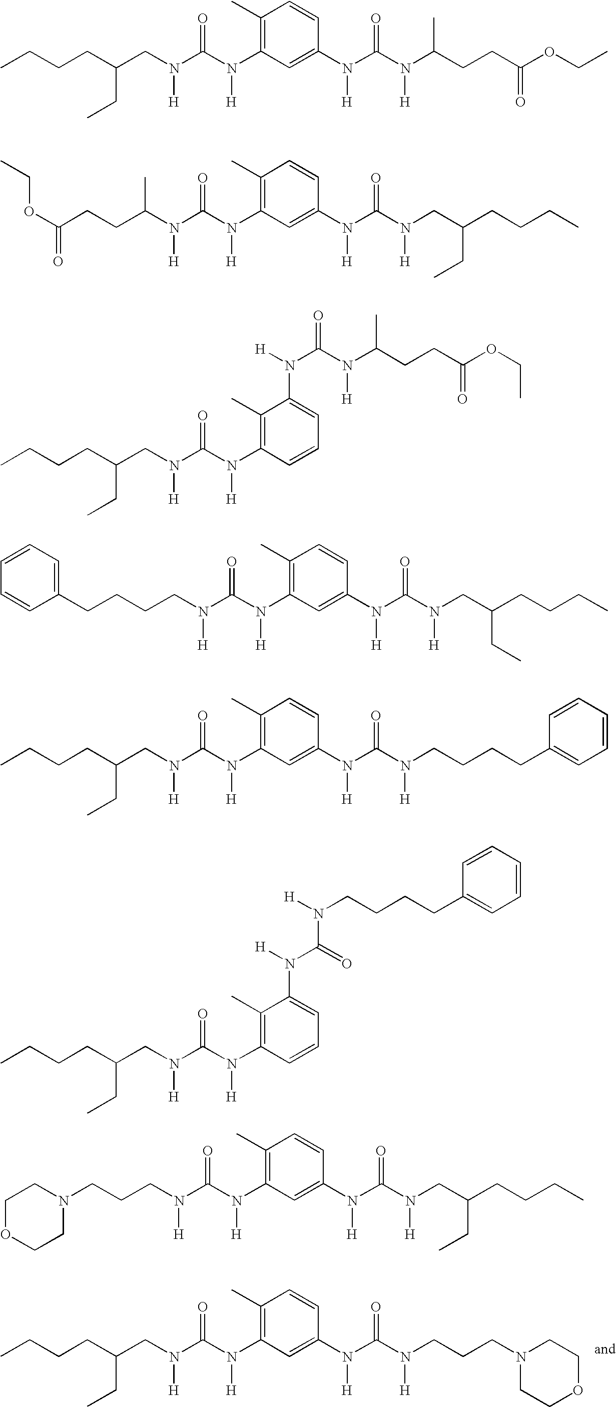

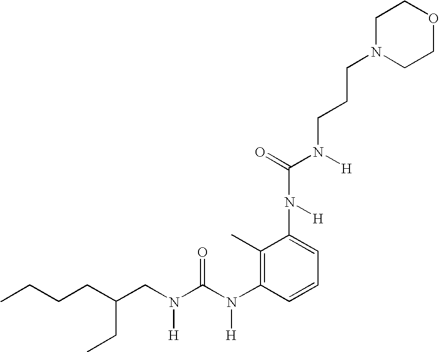

The expression “compounds that may be used in compositions according to the present disclosure” refers to a compound of formula (I) or (II), alone or as a mixture in various proportions, which may dissolve at room temperature in several cosmetic oils and which may be effective for gelling the oil or oil mixture under consideration, for example giving it desired physical and/or chemical properties.

For the purposes of the present disclosure, the term “effective amount” denotes an amount that is sufficient to obtain texturing of the oil or oil mixture under consideration in the composition according to the present disclosure.

This texturing may be reflected by an increase in the viscosity, for example due to the introduction of at least one compound of formula (II).

For example, compositions according to the present disclosure may comprise from 0.0001% to 5% by weight of an asymmetric bis-urea of formula (II), such as from 0.001% to 1%, for example from 0.004% to 0.5% by weight of at least one asymmetric bis-urea of formula (II) relative to the total weight of the liquid fatty phase.

The effective amount may vary depending, for example, on the nature of the substituents R₁ and/or R₂ of the bis-urea derivative, the positional isomer, and whether or not it is used in pure form or as a mixture with other bis-urea derivatives of formula (II), and also on the nature of the oily phase.

In one embodiment of the present disclosure, the asymmetric bis-urea derivatives of formula (II) may be used in the form of a mixture of derivatives of formula (II) with each other and/or with the two corresponding forms of symmetric bis-urea derivatives. For the purposes of the present disclosure, the term “corresponding forms of symmetric bis-urea derivatives” means the bis-urea derivatives according to formula (II) with identical radicals R₁ and the bis-urea derivatives according to formula (II) with identical radicals R₂.

In some embodiments of the present disclosure, the compound of formula (II) may be derived from the reaction between at least one diisocyanate of formula:

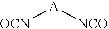

and at least two different primary amines of formulae:

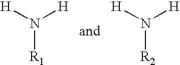

wherein A, R₁ and R₂ are defined as above.

In some embodiments of the present disclosure, the various diisocyanates may be positional isomers of the substituent R₃ on the group A, for example in 95/5 or 80/20 proportions.

The term “at least two different primary amines” means that other primary amines Rx—NH₂ in which Rx is chosen from the definitions proposed for R₁ and R₂, may be added thereto.

In one embodiment of the present disclosure, the number of amines used for the reaction may be greater than or equal to 2 and may range, for example, from 2 to 20, for example, from 3 and 10. To facilitate the preparation of the composition and the characterization of the mixture thus obtained, it may be advantageous to remain limited to the use of two amines.

In one embodiment of the present disclosure, the amines used are, taken together, in a mole ratio ranging from 2 to 3 equivalents, for example, from 2.1 to 2.5, such as 2.2 equivalents of amines, per one equivalent of diisocyanate(s).

In one embodiment wherein only two primary amines are used for the reaction, the mole ratio n(R₁)/n(R₂) may range from 1/99 and 99/1, for example, from 5/95 and 95/5, for example, from 10/90 and 90/10, with n(R₁) corresponding to the number of moles of:

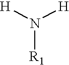

and n(R₂) corresponding to the number of moles of:

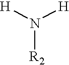

and wherein R₁ and R₂ are defined as above.

The reaction may be performed under an inert atmosphere, for example under argon, and/or in an anhydrous medium. The reaction medium temperature may be maintained below 50° C., for example ranging from 15° C. to 40° C., for example ranging from 18° C. to 25° C.

The diisocyanate(s) may be dissolved in an anhydrous solvent such as, but not limited to, tetrahydrofuran, 2-methyltetrahydrofuran, N-methylpyrrolidone, butyl acetate or methyl ethyl ketone. The concentration of the diisocyanate(s) in the anhydrous solvent may range from 1% to 30% by weight, for example from 2% to 20%, such as from 4% to 10% by weight.

A solution comprising the amines may be prepared in the same solvent as the diisocyanate(s) to a concentration ranging, for example, from 0.1% to 99.9% by mass. In one embodiment, the temperature of the reaction medium does not exceed 40° C. and the amine concentration and the rate of addition of the solution comprising the amines can be adjusted to this need. The reaction medium may be left stirring, for example, for 30 minutes to 12 hours. The reaction progress may be monitored by infrared spectrometry (for example by observing the disappearance of the NCO band between 2250 and 2280 cm⁻¹). For example, at the end of the reaction, the reaction medium may be poured into a large amount of acidic water (for example, water adjusted to pH 3-4 with HCl). A precipitate can then be obtained, which is filtered off, washed, for example several times, for example with water, and dried under reduced pressure, for example under vacuum or freeze-dried.

After this reaction, at least one of the following two forms:

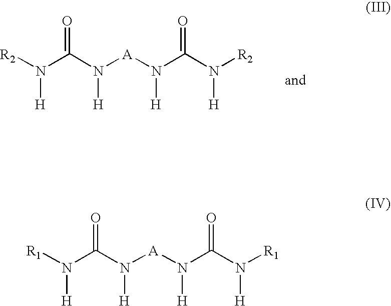

wherein A, R₁ and R₂ are defined as above, with A being identical in formulae (II), (III) and (IV), R₁ being identical in formulae (II) and (IV), and R₂ being identical in formulae (II) and (III),
can be obtained together with the expected asymmetric derivative of formula (II).

The precipitate comprises the compounds of formula (II), and may be characterized by NMR spectrometry (such as ¹H and/or ¹³C), and/or by HPLC, and may be used in its native form for texturing the oily medium under consideration.

At the end of the reaction, the mixture of bis-ureas of formulae (II), (III) and (IV) may be isolated and may be used in its native form for gelling the desired oily medium.

In this case, the compound of formula (II) may be used in the liquid fatty phase in the form of a mixture with the compounds of formulae (III) and (IV).

In one embodiment, the mixture of bis-ureas may be soluble in the liquid fatty phase to be textured at a temperature of less than or equal to 50° C., for example less than or equal to 30° C., for example at room temperature.

### Hydrocarbonated Bis-Urea Compatible Oils

Compositions according to the present disclosure may comprise at least one continuous liquid fatty phase comprising at least one non-silicone oil with a solubility parameter δₐ ranging from 0 to 5.00 (J/cm³)^{1/2}, for example ranging from 0 to 4.00 (J/cm³)^{1/2}.

For the purposes of the present disclosure, the term “non-silicone oil” refers to an oil not comprising any silicon atoms.

In one embodiment of the present disclosure, the non-silicone oil is non-volatile.

For the purposes of the present patent application, the term “non-volatile oil” refers to an oil, organic solvent, or non-aqueous medium that does not evaporate (no loss of mass) when it is placed in contact with the skin for one hour at room temperature and atmospheric pressure. In one embodiment, the non-volatile oil is an oil that is liquid at room temperature, for example having a vapor pressure of less than 0.01 mmHg (1.33 Pa) at room temperature and pressure.

The solubility parameter δₐ is calculated by the relationship δₐ=(δₚ²+δₕ²)^{1/2} in which the parameters δₚ and δₕ correspond to the Hansen solubility parameters:
δₚ characterizes the Debye forces of interaction between permanent dipoles;
δₕ characterizes specific interaction forces (such as hydrogen bonding, acid/base, donor/acceptor, etc).

The parameters δₚ and δₕ may be expressed in (J/cm³)^{1/2}. They are determined at room temperature (25° C.) and in particular according to the calculation method indicated in patent JP-A-08-109 121.

The definition of the Hansen solubility parameters is well known to those skilled in the art, and is described in the article by C. M. Hansen: “The three-dimensional solubility parameters”, J. Paint Technol., 39, 105 (1967). These parameters are also described in the Kao document JP-A-08-109 121 and the D. W. Van Krevelen document “Properties of polymers” (1990), p. 190.

In one embodiment of the present disclosure, the non-silicone oils with a solubility parameter δₐ ranging from 0 to 5.00 (J/cm³)^{1/2} may be chosen from squalane, parleam oil, polyisobutylene, jojoba oil, sesame oil, isopropyl myristate, butyl stearate, isononyl isononanoate, isopropyl palmitate, arachidyl propionate, stearyl heptanoate, isopropyl stearate, isostearyl neopentanoate, 2-ethylhexyl palmitate, cetyl 2-ethylhexanoate, isopropyl isostearate, C₁₂-C₁₅ alkyl benzoate, macadamia oil, octyldodecyl stearoylstearate, arara oil, PVP/hexadecene copolymer, oleyl erucate, octyldodecyl stearate, isostearyl palmitate, isocetyl stearate, didecene, hydrogenated polydecene, diisocetyl dodecanedioate, isostearyl isostearate, dicaprylyl ether, pentaerythrityl tetraisostearate, glyceryl triisostearate, octyldodecyl neopentanoate, isoeicosane, diisopropyl dimer dilinoleate, dioctyidodecyl dimer dilinoleate, octyldodecyl myristate, tridecyltetradecanoin, triisostearyl trilinoleate, isostearyl benzoate, isodecyl isononanoate, diisostearyl adipate, tridecyl isononanoate, triisopalmitine, 2-decyl hexyl isononanoate, 2-octyldodecyl benzoate, diisoarachidyl dodecanedioate, propylene glycol diisostearate, hydrogenated dimer dilinoleyl/dimethyl carbonate copolymer, octyidodecyl neodecanoate, octyldodecyl octanoate, isostearyl isononanoate, isofol-24 isostearate, octyldodecyl erucate, pentaerythrityl tetraoctyldodecanoate, pentaerythrityl tetradecyltetradecanoate, pentaerythrityl tetrakis(2-hexyldecanoate), ditrimethylol-propane tetraisostearate, castor oil benzoate (ratio 1/1.5), octyidodecyl PPG-3 myristyl ether dimer dilinoleate and trimethylolpropane triisostearate, and mixtures thereof.

In one embodiment, the non-silicone oil with a solubility parameter δₐ ranging from 0 to 5.00 (J/cm³)^{1/2} is non-volatile.

In one embodiment, the non-silicone oil with a solubility parameter δₐ ranging from 0 to 5.00 (J/cm³)^{1/2} is aprotic.

The term “aprotic oil” herein refers to an oil comprising few or no hydrogen atoms bonded to a highly electronegative atom such as O or N.

For example, the term “aprotic oil” may refer to oils that comprise, as a function of the yield of their synthesis, residual groups bearing a labile hydrogen atom (for example OH, NH, and/or COOH residual groups) in a content of less than or equal to 5% by number.

In one embodiment, the non-silicone oil with a solubility parameter δₐ ranging from 0 to 5.00 (J/cm³)^{1/2} is chosen from alkanes, esters, ethers and carbonates, and mixtures thereof.

In one embodiment, the non-silicone oil with a solubility parameter δₐ ranging from 0 to 5.00 (J/cm³)^{1/2} is chosen from alkanes comprising from 14 to 65 carbon atoms, for example from 22 to 35 carbon atoms.

In one embodiment, the non-silicone oil with a solubility parameter δₐ ranging from 0 to 5.00 (J/cm³)^{1/2} may be chosen from squalane, polyisobutylenes with a molecular weight ranging from 250 to 800 g/mol, such as parleam oil, and isoeicosane, and mixtures thereof.

In one embodiment, the non-silicone oil with a solubility parameter δₐ ranging from 0 to 4.00 (J/cm³)^{1/2} is parleam oil.

In one embodiment, the non-silicone oil with a solubility parameter δₐ ranging from 0 to 5.00 (J/cm³)^{1/2} is chosen from esters, ethers, and carbonates, and mixtures thereof, with a molecular mass ranging from 250 to 3000 g/mol.

Such esters, ethers, and carbonates may be chosen from jojoba oil, sesame oil, isopropyl myristate, butyl stearate, isononyl isononanoate, isopropyl palmitate, isoeicosane, arachidyl propionate, stearyl heptanoate, isopropyl stearate, isostearyl neopentanoate, 2-ethylhexyl palmitate, cetyl 2-ethylhexanoate, isopropyl isostearate, C₁₂-C₁₅ alkyl benzoate, macadamia oil, octyldodecyl stearoylstearate, arara oil, oleyl erucate, octyldodecyl stearate, isostearyl palmitate, isocetyl stearate, diisocetyl dodecanedioate, isostearyl isostearate, dicaprylyl ether, pentaerythrityl tetraisostearate, glyceryl triisostearate, octyldodecyl neopentanoate, diisopropyl dimer dilinoleate, dioctyldodecyl dimer dilinoleate, octyldodecyl myristate, tridecyltetradecanoin, triisostearyl trilinoleate, isostearyl benzoate, isodecyl isononanoate, diisostearyl adipate, tridecyl isononanoate, triisopalmitine, 2-hexyldecyl isononanoate, 2-octyldodecyl benzoate, diisoarachidyl dodecanedioate, propylene glycol diisostearate, hydrogenated dimer dilinoleyl/dimethyl carbonate copolymer, octyldodecyl neodecanoate, octyidodecyl octanoate, isostearyl isononanoate, isofol-24 isostearate, octyldodecyl erucate, pentaerythrityl tetraoctyldodecanoate, pentaerythrityl tetradecyltetradecanoate, pentaerythrityl tetrakis(2-hexyldecanoate), ditrimethylolpropane tetraisostearate, castor oil benzoate (ratio 1/1.5), octyidodecyl PPG-3 myristyl ether dimer dilinoleate and trimethylolpropane triisostearate, and mixtures thereof.

In one embodiment, the non-silicone oil with a solubility parameter δₐ ranging from 0 to 5.00 (J/cm³)^{1/2} may be present in compositions according to the present disclosure in an amount ranging from 20% to 100% by weight, for example from 40% to 99% by weight, for example from 60% to 95% by weight, relative to the total weight of the continuous liquid fatty phase.

### Additional Oils:

In addition to the oils defined above, compositions according to the present disclosure may comprise at least one additional oil which is different from the oil described above and which may be chosen from volatile oils, non-volatile oils, and mixtures thereof.

In one embodiment, compositions according to the present disclosure may comprise at least one volatile oil.

For the purposes of the present disclosure, the term “volatile oil” refers to any oil that is capable of evaporating on contact with the skin at room temperature and atmospheric pressure. In one embodiment, the volatile oil is chosen from volatile cosmetic oils that are liquid at room temperature, with a non-zero vapor pressure at room temperature and atmospheric pressure, for example ranging from 0.13 Pa to 40 000 Pa (0.001 to 300 mmHg), for example ranging from 1.3 Pa to 1300 Pa (0.01 to 10 mmHg).

In one embodiment, the volatile oil may be chosen from volatile hydrocarbon-based oils, volatile silicone oils, volatile fluoro oils, and mixtures thereof.

In one embodiment, compositions according to the present disclosure may comprise at least one hydrocarbon-based volatile oil.

The term “hydrocarbon-based oil” refers to an oil mainly comprised of hydrogen and carbon atoms and possibly oxygen, nitrogen, sulfur, and/or phosphorus atoms.

In one embodiment, the volatile hydrocarbon-based oils may be chosen from hydrocarbon-based oils comprising from 8 to 16 carbon atoms, for example branched C₈-C₁₆ alkanes, for instance C₈-C₁₆ isoalkanes of petroleum origin (also known as isoparaffins), such as isododecane (also known as 2,2,4,4,6-pentamethyl-heptane), isohexadecane, and, for example, the oils sold under the trade names Isopar® and Permethyl®.

In one embodiment, the volatile oils may be chosen from volatile silicones, for instance volatile linear or cyclic silicone oils, for example those with a viscosity less than or equal to 5 centistokes (5×10⁻⁶ m²/s), and/or for example those comprising from 2 to 10 silicon atoms, for instance from 2 to 7 silicon atoms, these silicones optionally comprising alkyl or alkoxy groups comprising from 1 to 10 carbon atoms. As volatile silicone oils that may be used in compositions according to the present disclosure, non-limiting mention may be made of octamethylcyclotetrasiloxane, decamethylcyclopentasiloxane, dodecamethylcyclohexasiloxane, hepta-methylhexyltrisiloxane, heptamethyloctyltrisiloxane, hexamethyldisiloxane, octamethyltrisiloxane, decamethyltetrasiloxane and dodecamethylpentasiloxane, and mixtures thereof.

In one embodiment, the volatile fluoro oil does not have a flash point.

As volatile fluoro oils that may be used in compositions according to the present disclosure, non-limiting mention may be made of nonafluoroethoxybutane, nonafluoromethoxybutane, decafluoropentane, tetradecafluorohexane, dodecafluoro-pentane, and mixtures thereof.

Compositions according to the present disclosure may comprise a volatile oil in an amount ranging from 1% to 50% by weight, for example from 2% to 30% by weight, for example from 3% to 15% by weight, relative to the total weight of the composition.

Compositions according to the present disclosure may comprise at least one non-volatile oil.

The term “non-volatile oil” refers to an oil that remains on the skin at room temperature and atmospheric pressure for at least several hours, for example, an oil that has a vapor pressure of less than 0.13 Pa (0.01 mmHg).

These non-volatile oils may be hydrocarbon-based oils of animal or plant origin, or silicone oils, or mixtures thereof. The term “hydrocarbon-based oil” refers to an oil mainly comprised of hydrogen and carbon atoms and possibly oxygen, nitrogen, sulfur, and/or phosphorus atoms.

The non-volatile oils may be chosen from hydrocarbon-based oils, which may be fluorinated, and/or non-volatile silicone oils.

As non-volatile hydrocarbon-based oils that may be used in compositions according to the present disclosure, non-limiting mention may be made of:
hydrocarbon-based oils of animal origin,
hydrocarbon-based oils of plant origin, such as triglycerides comprising of fatty acid esters of glycerol, the fatty acids of which may have varied chain lengths from C₄ to C₂₄, these chains possibly being linear or branched, and saturated or unsaturated; for example, these oils can be heptanoic or octanoic acid triglycerides; or wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil and musk rose oil; shea butter; or caprylic/capric acid triglycerides, for instance those sold by the company Stearineries Dubois or those sold under the names Miglyol 810®, 812® and 818® by the company Dynamit Nobel; and mixtures thereof,
synthetic ethers comprising from 10 to 40 carbon atoms,
linear or branched hydrocarbons of mineral or synthetic origin, such as petroleum jelly, polydecenes, hydrogenated polyisobutene such as Parleam®, squalane, and liquid paraffins, and mixtures thereof,
synthetic esters, for instance oils of formula R₁COOR₂ in which R₁ represents a linear or branched fatty acid residue comprising from 1 to 40 carbon atoms, R₂ represents a hydrocarbon-based chain, which can be branched, comprising from 1 to 40 carbon atoms, and R₁+R₂≧10, such as purcellin oil (cetostearyl octanoate), isopropyl myristate, isopropyl palmitate, C₁₂ to C₁₅ alkyl benzoates, hexyl laurate, diisopropyl adipate, isononyl isononanoate, isodecyl neopentanoate, 2-ethylhexyl palmitate, isostearyl isostearate, 2-hexyldecyl laurate, 2-octyldecyl palmitate, 2-octyidodecyl myristate, and alcohol or polyalcohol heptanoates, octanoates, decanoates or ricinoleates, such as propylene glycol dioctanoate; hydroxylated esters, for instance isostearyl lactate, diisostearyl malate, and 2-octyldodecyl lactate; polyol esters and pentaerythritol esters; and mixtures thereof,
fatty alcohols that are liquid at room temperature with a branched and/or unsaturated carbon-based chain comprising from 12 to 26 carbon atoms, such as octyidodecanol, isostearyl alcohol, oleyl alcohol, 2-hexyldecanol, 2-butyloctanol, 2-undecylpentadecanol, and mixtures thereof,
higher fatty acids such as oleic acid, linoleic acid, and linolenic acid, and mixtures thereof.

The non-volatile silicone oils that may be used in compositions according to the present disclosure may be non-volatile polydimethylsiloxanes (PDMS); poly-dimethylsiloxanes comprising alkyl or alkoxy groups, which are pendant and/or at the end of a silicone chain, these groups each comprising from 2 to 24 carbon atoms; phenyl silicones, for instance phenyl trimethicones; phenyl dimethicones; phenyltrimethylsiloxydiphenylsiloxanes; diphenyl dimethicones; diphenylmethyl-diphenyltrisiloxanes; and mixtures thereof.

The non-volatile oil may be present in compositions according to the present disclosure in an amount ranging from 1% to 50% by weight, for example ranging from 2% to 30% by weight, for example ranging from 3% to 20% by weight, relative to the total weight of the composition.

The liquid fatty phase may be present in compositions according to the present disclosure in an amount ranging from 10% to 95% by weight, for example ranging from 20% to 90% by weight, for example ranging from 30% to 85% by weight, relative to the total weight of the composition.

### Fatty Substances

Compositions according to the present disclosure may also comprise fatty substances in addition to the oils mentioned above, for example, but not limited to, waxes and/or pasty fatty substances.

The term “waxes” refers to a fatty substance that is solid at room temperature.

The term “pasty fatty substances” refers to substances having at least one of the following physicochemical properties:
a viscosity ranging from 0.1 to 40 Pa·s (1 to 400 poises), measured at 40° C. with a Contraves TV rotary viscometer equipped with a MS-r3 or MS-r4 spindle at a frequency of 60 Hz,
a melting point ranging from 25 to 70° C., for example ranging from 25 to 55° C.

As waxes that may be used in compositions according to the present disclosure, non-limiting mention may be made of:
waxes of animal origin such as beeswax, spermaceti, lanolin wax, and lanolin derivatives, plant waxes such as carnauba wax, candelilla wax, ouricury wax, Japan wax, cocoa butter, cork fiber wax, and sugar cane wax, and mixtures thereof,
mineral waxes, for example paraffin wax, petroleum jelly wax, lignite wax, microcrystalline waxes, ozokerites, and mixtures thereof,
synthetic waxes, for example polyethylene waxes, waxes obtained by Fisher-Tropsch synthesis, and mixtures thereof,
silicone waxes, such as substituted linear polysiloxanes; for example, polyether silicone waxes, alkyl and alkoxy dimethicones comprising from 16 to 45 carbon atoms, and alkyl methicones, for instance the C₃₀-C₄₅ alkyl methicone sold under the trade name AMS C30 by Dow Corning, and mixtures thereof,
hydrogenated oils that are solid at 25° C., such as hydrogenated castor oil, hydrogenated jojoba oil, hydrogenated palm oil, hydrogenated tallow, hydrogenated coconut oil, fatty esters that are solid at 25° C., for instance the C₂₀-C₄₀ alkyl stearate sold under the trade name Kester Wax K82H by the company Koster Keunen, and mixtures thereof,
and/or mixtures thereof.

In one embodiment of the present disclosure, polyethylene waxes, microcrystalline waxes, carnauba waxes, hydrogenated jojoba oil, candelilla waxes and beeswaxes, and/or mixtures thereof, can be used.

Without wishing to be limited by any theory, waxes may make it possible to reinforce the cicatrization properties and/or to reduce the tacky to pasty nature of compositions according to the present disclosure.

In one embodiment of the present disclosure, waxes may be present in an amount ranging from 0.1% to 30% by weight, for example from 0.5% to 20% by weight, relative to the total weight of the composition.

These fatty substances may be chosen in a varied manner by a person skilled in the art in order to prepare compositions having the desired properties, for example in terms of consistency or texture.

### Additional Thickener

In addition to bis-urea compounds as described previously, compositions according to the present disclosure may comprise at least one additional oil thickener chosen from polymeric thickeners and mineral thickeners, and mixtures thereof.

The polymeric oil thickener may be capable of thickening or gelling the organic phase of the composition. The polymeric thickener may also be film-forming, i.e. it may be capable of forming a film during its application to the skin.

Polymeric oil thickeners may be chosen from:
polycondensates of polyamide type resulting from the condensation of (a) at least one acid chosen from dicarboxylic acids comprising at least 32 carbon atoms, such as fatty acid dimers, and (b) at least one alkylenediamine, for example ethylenediamine; in which the polycondensate of polyamide type comprises at least one carboxylic acid end group esterified or amidated with at least one saturated, linear monoalcohol or one saturated, linear monoamine comprising from 12 to 30 carbon atoms, for example ethylenediamine/stearyl dilinoleate copolymers such as the product sold under the name Uniclear 100 VG® by the company Arizona Chemical;
silicone polymers such as:

1) polyorganosiloxanes comprising at least two groups capable of hydrogen bonding interactions, these two groups being located in the polymer chain, and/or

2) polyorganosiloxanes comprising at least two groups capable of hydrogen bonding interactions, these two groups being located on grafts to or branches of the polymer chain.

As groups capable of hydrogen bonding interactions, non-limiting mention may be made of ester, amide, sulfonamide, carbamate, thiocarbamate, urea, urethane, thiourea, oxamido, guanidino and biguanidino groups, and combinations thereof.

As silicone polymers which can be used as structuring agents in compositions according to the present disclosure, non-limiting mention may be made of polymers of the polyorganosiloxane type, for instance those described in the following U.S. Pat. Nos. 5,874,069; 5,919,441; 6,051,216; and 5,981,680.

In one embodiment of the present disclosure, the silicone polymers can be polyorganosiloxanes as defined above in which the groups capable of hydrogen bonding interactions are located in the polymer chain.

In one embodiment of the present disclosure, the silicone polymers may be polymers comprising at least one group corresponding to the formula V:

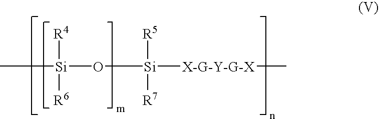

in which:

1) R⁴, R⁵, R⁶ and R⁷, which may be identical or different, represent groups chosen from:
linear, branched and cyclic, saturated or unsaturated, C₁ to C₄₀ hydrocarbon-based groups, optionally comprising in their chain one or more oxygen, sulfur and/or nitrogen atoms, and optionally being partially or totally substituted with fluorine atoms,
C₆ to C₁₀ aryl groups, optionally substituted with one or more C₁ to C₄ alkyl groups,
polyorganosiloxane chains optionally comprising one or more oxygen, sulfur and/or nitrogen atoms;

2) the groups X, which may be identical or different, represent linear or branched C₁ to C₃₀ alkylenediyl groups, optionally comprising in their chain one or more oxygen and/or nitrogen atoms;

3) Y represents a saturated or unsaturated, linear or branched, C₁ to C₅₀ divalent alkylene, arylene, cycloalkylene, alkylarylene, or arylalkylene group, optionally comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl, and C₁ to C₆ aminoalkyl groups; or

4) Y represents a group corresponding to the formula:

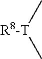

in which
T represents a linear or branched, saturated or unsaturated, C₃ to C₂₄ trivalent or tetravalent hydrocarbon-based group, optionally substituted with a polyorganosiloxane chain, and optionally comprising one or more atoms chosen from O, N, and S; or T represents a trivalent atom chosen from N, P, and Al; and
R⁸ represents a linear or branched C₁ to C₅₀ alkyl group or a polyorganosiloxane chain, optionally comprising one or more ester, amide, urethane, thiocarbamate, urea, thiourea and/or sulfonamide groups, which may optionally be linked to another chain of the polymer;

5) the groups G, which may be identical or different, represent divalent groups chosen from:

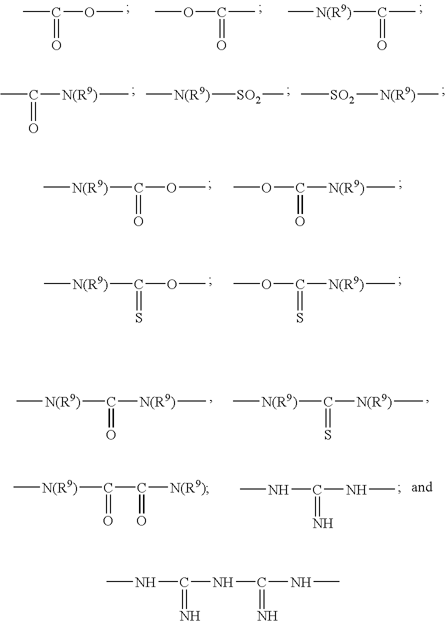

in which R⁹ represents a hydrogen atom or a linear or branched C₁ to C₂₀ alkyl group, wherein at least 50% of the groups R⁹ of the polymer represent a hydrogen atom and at least two of the groups G of the polymer are a group other than:

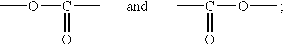

6) n is an integer ranging from 2 to 500, for example from 2 to 200, and m is an integer ranging from 1 to 1000, for example from 1 to 700, for example from 6 to 200.

In one embodiment of the present disclosure, 80% of the groups R⁴, R⁵, R⁶, and R⁷ of the polymer may be chosen from methyl, ethyl, phenyl, and 3,3,3-trifluoropropyl groups.

In one embodiment of the present disclosure, the groups capable of hydrogen bonding interactions are amide groups of formulae —C(O)NH— and —HN—C(O)—.

In this case, the structuring agent may be chosen from polymers comprising at least one unit of formula (VI) or (VII):

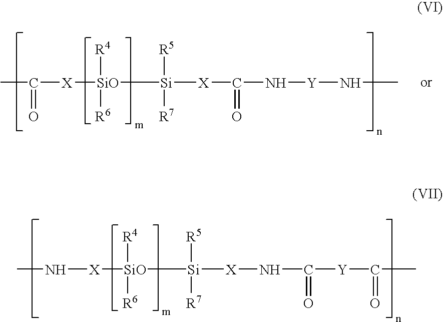

in which R⁴, R⁵, R⁶, R⁷, X, Y, m and n are as defined above.

In these polyamides of formula (VI) or (VII),
m may range from 1 to 700, for example from 15 to 500, for example from 50 to 200, and n may range from 1 to 500, for example from 1 to 100, for example from 4 to 25,
X may be chosen from linear and branched alkylene chains comprising from 1 to 30 carbon atoms, from example from 1 to 20 carbon atoms, for example from 5 to 15 carbon atoms, for example 10 carbon atoms, and
Y may be chosen from alkylene chains that are linear or branched and/or that possibly comprise rings and/or unsaturations, comprising from 1 to 40 carbon atoms, for example from 1 to 20 carbon atoms, for example from 2 to 6 carbon atoms, for example 6 carbon atoms.

In one embodiment of the present disclosure, the structuring agent may be chosen from galactomannans comprising from one to six, for example from two to four, hydroxyl groups per saccharide, optionally substituted with a saturated or unsaturated alkyl chain, for instance guar gum alkylated with C₁-C₆, for example C₁-C₃, alkyl chains, and mixtures thereof.

Compositions according to the present disclosure may also comprise at least one mineral oil thickener such as an organophilic clay or fumed silicas.

The term “organophilic clays” refers to clays modified with chemical compounds such that the clay is able to swell in oily media.

Clays are products that are already known per se, and which are described, for example, in the publication “Mineralogie des argues [Mineralogy of clays], S. Caillére, S. Hénin, M. Rautureau, 2nd Edition 1982, Masson”, the teachings of which are included herein by way of reference.

Clays may be silicates comprising a cation that may be chosen from calcium, magnesium, aluminum, sodium, potassium, and lithium cations, and mixtures thereof.

As clays which may be used in compositions according to the present disclosure, non-limiting mention may be made of clays of the smectite family such as montmorillonites, hectorites, bentonites, beidellites, and saponites, and also clays of the vermiculite, stevensite, and chlorite families, and mixtures thereof.

These clays may be of natural or synthetic origin. In one embodiment of the present disclosure, clays that are cosmetically compatible and/or compatible with keratin materials such as the skin are used.

The organophilic clay may be chosen from montmorillonite, bentonite, hectorite, attapulgite, and sepiolite, and mixtures thereof. In one embodiment of the present disclosure, a bentonite and/or a hectorite may be used.

The organophilic clays may be modified with a chemical compound chosen from quaternary amines, tertiary amines, amine acetates, imidazolines, amine soaps, fatty sulfates, alkyl aryl sulfonates, and amine oxides, and mixtures thereof.

As organophilic clays that may be used in compositions according to the present disclosure, non-limiting mention may be made of quaternium-18 bentonites such as those sold under the names Bentone 3, Bentone 38, and Bentone 38V by the company Rheox, Bentone ISD V by the company Elementis, Tixogel VP by the company United Catalyst, and Claytone 34, Claytone 40, and Claytone XL by the company Southern Clay; stearalkonium bentonites such as those sold under the names Bentone 27 by the company Rheox, Tixogel LG by the company United Catalyst, and Claytone AF and Claytone APA by the company Southern Clay; and quaternium-18/benzalkonium bentonites such as those sold under the names Claytone HT and Claytone PS by the company Southern Clay.

Fumed silicas may be obtained by high-temperature hydrolysis of a volatile silicon compound in an oxhydric flame, producing a finely divided silica. This process may make it possible to obtain hydrophilic silicas having a large number of silanol groups at their surface. Such hydrophilic silicas are sold, for example, under the names Aerosil 130®, Aerosil 200®, Aerosil 255®, Aerosil 300®, and Aerosil 380® by the company Degussa, and Cab-O-Sil HS-5®, Cab-O-Sil EH-5®, Cab-O-Sil LM-130®, Cab-O—Sil MS-55®, and Cab-O-Sil M-5® by the company Cabot.

The surface of the said silica may be chemically modified, such as via a chemical reaction resulting in a reduction in the number of silanol groups. For example, it is possible to substitute silanol groups with hydrophobic groups: a hydrophobic silica is then obtained.

As hydrophobic groups, non-limiting mention may be made of:
trimethylsiloxyl groups, for example those obtained by treating fumed silica in the presence of hexamethyldisilazane. Silicas thus treated may be known as “silica silylate” according to the CTFA (6th Edition, 1995). Such silicas are sold, for example, under the references Aerosil R8120 by the company Degussa and Cab-O-Sil TS-530® by the company Cabot;
dimethylsilyloxyl or polydimethylsiloxane groups, for example those obtained by treating fumed silica in the presence of polydimethylsiloxane or dimethyldichlorosilane. Silicas thus treated may be known as “silica dimethyl silylate” according to the CTFA (6th Edition, 1995). Such silicas are sold, for example, under the references Aerosil R972® and Aerosil R974® by the company Degussa and Cab-O-Sil TS-610® and Cab-O-Sil TS-720® by the company Cabot.

In one embodiment, the fumed silica has a particle size that may be nanometric to micrometric, for example ranging from about 5 to 200 nm.

Without wishing to be limited by any theory, the mineral thickeners may make it possible to reinforce the cicatrization properties and/or to reduce the tacky to pasty nature of the compositions according to the present disclosure.

The additional oil thickener may be present in compositions according to the present disclosure in an amount ranging from 0.01% to 15% by weight, for example from 0.1% to 10% by weight, for example from 0.3% to 5% by weight, relative to the total weight of the composition.

### Aqueous Phase

Compositions according to the present disclosure may comprise an aqueous phase.

The aqueous phase comprises water. For example, the water may be a floral water such as cornflower water, and/or a mineral water such as eau de Vittel, eau de Lucas, or eau de La Roche Posay, and/or a spring water, or a mixture thereof.

The aqueous phase may also comprise organic solvents that are water-miscible (at room temperatures, for example, at 25° C.), for example monoalcohols comprising from 2 to 6 carbon atoms such as ethanol or isopropanol; polyols comprising from 2 to 20 carbon atoms, for example comprising from 2 to 10 carbon atoms, for example comprising from 2 to 6 carbon atoms, such as glycerol, propylene glycol, butylene glycol, pentylene glycol, hexylene glycol, dipropylene glycol, diethylene glycol, and mixtures thereof; glycol ethers (for example comprising from 3 to 16 carbon atoms), such as C₁-C₄ alkyl ethers of monopropylene, dipropylene, or tripropylene glycol, and C₁-C₄ alkyl ethers of monoethylene, diethylene, or triethylene glycol, and mixtures thereof.

The aqueous phase may also comprise stabilizers, for example sodium chloride, magnesium dichloride, or magnesium sulfate.

The aqueous phase may also comprise any water-soluble or water-dispersible compound that is compatible with an aqueous phase, for example gelling agents, film-forming agents, thickeners, surfactants, and mixtures thereof.

In one embodiment, the aqueous phase may be present in compositions according to the present disclosure in an amount ranging from 1% to 80% by weight, for example from 5% to 50% by weight, for example from 5% to 40% by weight, relative to the total weight of the composition.

In one embodiment, compositions according to the present disclosure may also be anhydrous. For the purposes of the present patent application, the term “anhydrous composition” refers to a composition comprising less than 5% by weight of water, for example less than 2% by weight of water, relative to the total weight of the composition. In one embodiment, compositions according to the present disclosure may be substantially free of water, water not being added during the preparation of the composition, but corresponding to the residual water provided by the mixed ingredients.

In one embodiment, the aqueous phase and the oily phase may be present in an amount such that the weight ratio of water to oil is greater than or equal to 1, for example ranging from 1 to 1.2.

### Pulverulent Phase

Compositions according to the present disclosure may comprise a pulverulent phase, for example a phase comprising pigments, fillers, and nacres, and/or mixtures thereof.

In one embodiment, compositions according to the present disclosure may comprise pigments.

The term “pigments” herein refers to mineral or organic particles which are insoluble in the liquid organic phase and which may color and/or opacify the composition.

Pigments used in compositions according to the present disclosure may be mineral or organic pigments. Pigments that may be used include, but are not limited to, metal oxides, for instance iron oxides (for example, yellow, red, brown, and black iron oxides), titanium dioxides, cerium oxide, zirconium oxide, and chromium oxide; manganese violet, ultramarine blue, Prussian blue, cobalt blue, and ferric blue; and mixtures thereof.

In one embodiment of the present disclosure, iron oxides or titanium oxide pigments can be used.

Pigments used in compositions according to the present disclosure may be treated with a hydrophobic agent to make them compatible with the organic phase of the composition. The hydrophobic agent may be chosen from silicones, for instance methicones, dimethicones, or perfluoroalkylsilanes; fatty acids, for instance stearic acid; metal soaps, for instance aluminum dimyristate, the aluminum salt of hydrogenated tallow glutamate, perfluoroalkyl phosphates, perfluoroalkylsilanes, perfluoroalkyl-silazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

N-acylamino acids used in compositions according to the present disclosure may comprise an acyl group comprising from 8 to 22 carbon atoms, for example a 2-ethylhexanoyl, caproyl, lauroyl, myristoyl, palmitoyl, stearoyl, or cocoyl group. The salts of these compounds may be aluminum, magnesium, calcium, zirconium, zinc, sodium, or potassium salts. The amino acid may be, for example, lysine, glutamic acid, or alanine.

In one embodiment, the term “alkyl” mentioned in the compounds mentioned above refers to an alkyl group comprising from 1 to 30 carbon atoms, for instance comprising from 5 to 16 carbon atoms.

As hydrophobic-treated pigments which may be used in compositions according to the present disclosure, non-limiting mention may be made of the compounds described in patent application EP-A-1 086 683.

Pigments may be present in compositions according to the present disclosure in an amount ranging from 0.1% to 40% by weight, for example ranging from 1% to 30% by weight, for example ranging from 5% to 15% by weight, relative to the total weight of the composition.

In one embodiment, the pulverulent phase of compositions according to the present disclosure may comprise fillers and/or nacres.

In one embodiment, compositions according to the present disclosure may comprise fillers.

The term “fillers” herein refers to colorless or white, mineral or synthetic, particles of any form, which are insoluble in the medium of the composition irrespective of the temperature at which the composition is manufactured.

Fillers used in compositions according to the present disclosure may be mineral or organic and of any form, for example platelet-shaped, spherical, or oblong, irrespective of the crystallographic form (for example lamellar, cubic, hexagonal, orthorhombic, etc.). As fillers which may be used in compositions according to the present disclosure, non-limiting mention may be made of talc, mica, silica, kaolin, polyamide (Nylon®) powders, poly-β-alanine powders, polyethylene powders, polymethyl methacrylates, polyurethane powders such as the powder of the copolymer of hexamethylene diisocyanate and of trimethylol hexyl lactone sold under the name Plastic Powder D-400 by the company Toshiki, tetrafluoroethylene polymer (Teflon®) powders, micronized wax particles, for example carnauba microwaxes such as those sold under the name MicroCare 350® by the company Micro Powders, microwaxes of synthetic wax such as those sold under the name MicroEase 114S® by the company Micro Powders, microwaxes consisting of a mixture of carnauba wax and of polyethylene wax, such as those sold under the names MicroCare 300® and 310® by the company Micro Powders, microwaxes consisting of a mixture of carnauba wax and of synthetic wax, such as those sold under the name MicroCare 325® by the company Micro Powders, polyethylene microwaxes such as those sold under the names MicroPoly 200®, 220®, 220L® and 250S® by the company Micro Powders, and those sold under the name Cerapure H5-C by the company Shamrock, or polypropylene microwaxes such as those sold under the name Mattewax by the company Micro Powders; lauroyllysine, starch, boron nitride, hollow polymer microspheres such as those of polyvinylidene chloride/acrylonitrile, for instance Expancel® (Nobel Industrie), powders of acrylic acid copolymers, silicone resin powders, for example silsesquioxane powders (such as, but not limited to, silicone resin powders described in patent EP 293795; for example Tospearls® from Toshiba), elastomeric polyorganosiloxane particles, precipitated calcium carbonate, magnesium carbonate, magnesium hydrogen carbonate, hydroxyapatite, hollow silica microspheres, glass or ceramic microcapsules, and metal soaps derived from organic carboxylic acids comprising from 8 to 22 carbon atoms, for example from 12 to 18 carbon atoms, for example zinc stearate, magnesium stearate, lithium stearate, zinc laurate or magnesium myristate; barium sulfate, and mixtures thereof.

In one embodiment, compositions according to the present disclosure may comprise a polytetrafluoroethylene (PTFE) powder.

Fillers may be present in compositions according to the present disclosure in an amount ranging from 0.1% to 30% by weight, for example ranging from 0.5% to 20% by weight, for example ranging from 0.8% to 10% by weight, relative to the total weight of the composition.

In one embodiment, the pulverulent phase of compositions according to the present disclosure may comprise nacres.

The term “nacres” herein refers to iridescent particles, for example those produced by certain mollusks in their shell or those synthesized, which are insoluble in the medium of the composition.

As nacres which may be used in compositions according to the present disclosure, non-limiting mention may be made of white nacreous pigments such as bismuth oxychloride, mica coated with titanium or with bismuth oxychloride, colored nacreous pigments such as titanium mica with iron oxides, titanium mica, for instance with ferric blue or with chromium oxide, or titanium mica with an organic pigment, for example a pigment as previously described herein, nacreous pigments based on bismuth oxychloride, and mixtures thereof.

### Additional Dyes

Compositions according to the present disclosure may further comprise dyes chosen from water-soluble and liposoluble dyes.

As water-soluble dyes, non-limiting mention may be made of beetroot juice, methylene blue, and caramel.

The term “liposoluble dyes” herein refers to organic compounds that are soluble in fatty substances such as oils.

As liposoluble dyes, non-limiting mention may be made of Sudan red, D&C Red No 17, D&C Green No 6, β-carotene, soybean oil, Sudan brown, D&C Yellow No 11, D&C Violet No 2, D&C Orange No 5, quinoline yellow, annatto and bromo acids, and mixtures thereof.

### Galenicals

Compositions according to the present disclosure may be in various galenical forms, for example anhydrous, or for example in the form of water-in-oil emulsions, or multiple emulsions with an oily continuous phase, and may be in the form of gels, creams, or suspensions, in compact or hot-cast form, or in the form of sticks.

Compositions according to the present disclosure may be in the form of a makeup composition, for example a complexion product such as a foundation, a makeup rouge, or an eyeshadow; a lip product such as a lipstick, a lip gloss, or a lipcare product; a concealer product; a blusher, a mascara, or an eyeliner; an eyebrow makeup product, a lip pencil, or an eye pencil; a nail product such as a nail varnish or a nailcare product; a body or hair makeup product (for example, a hair lacquer or mascara); a skincare protecting composition for the face, the neck, the hands, or the body, for example an anti-wrinkle, anti-fatigue, or anti-ageing composition, or a moisturizing or medicated composition; an antisun, sun-protective, after-sun, or artificial tanning composition; a hair composition, such as for hair dyeing, haircare, or hair hygiene, for styling or holding the hairstyle or for shaping the hair.

### Additional Common Cosmetic Ingredients

Compositions according to the present disclosure may comprise at least one other cosmetic ingredient, for example one chosen from antioxidants, fragrances, preserving agents, neutralizers, surfactants, sunscreens, vitamins, moisturizers, self-tanning compounds, anti-wrinkle active agents, emollients, hydrophilic or lipophilic active agents, free-radical scavengers, deodorants, sequestrants, and film-forming agents, and mixtures thereof.

The object of the present disclosure is presented in greater detail in the examples below.

### EXAMPLE 1

A foundation having the composition below was prepared:

| | | Concentration |
|---|---|---|
| PHASE | Name | (mass %) |
| A1 | Compound of formula (I) in the form of a mixture of the | 1.50 |
| | compounds: | |
| | 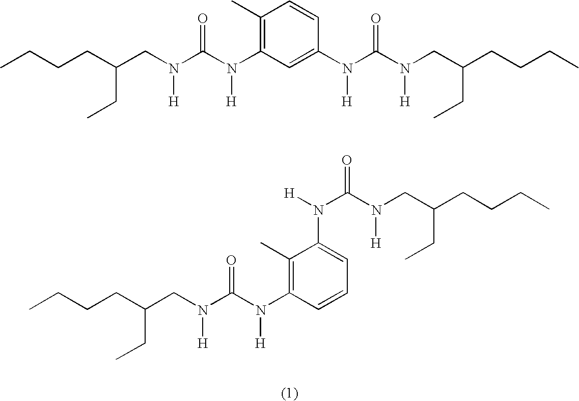 | |
| | Parleam oil | 32.10 |
| A2 | Cetyl dimethicone copolyol (2) | 1.80 |
| | Mono/diglycerides of isostearic acid esterified with | 0.60 |
| | succinic acid (3) | |
| A3 | Cyclopentasiloxane | 5.00 |
| | Iron oxide pigments | 2.04 |
| | Titanium dioxide pigments | 7.96 |
| A4 | Nylon 12 | 8.00 |
| B | Water | 40.00 |
| | Preserving agents | 1.00 |
| | TOTAL | 100.00 |

(1) The preparation of mixture (I) is described in the procedure below.
(2) Sold under the name Abil EM 90 by the company Goldschmidt.
(3) Sold under the name Imwitor 780 K by the company Sasol.

### Procedure:

1. Preparation of the Compound of Formula (I)

The following two compounds were provided:

(X)=50 g of tolylene diisocyanate (TDI) as a mixture of the 2,4 isomer and the 2,6 isomer in a 95/5 proportion,
(Y) ═79.6 g of 2-ethylhexylamine:

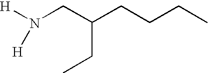

The mixture of tolylene diisocyanate dissolved in tetrahydrofuran (THF) with 2.2 equivalents of amine was prepared by reaction under argon. The reaction was performed under an inert atmosphere in anhydrous medium with a reaction medium temperature maintained between 15° C. and 40° C.

In parallel, a solution of amine (Y) in THF was prepared. Since the temperature of the reaction medium should not exceed 40° C., the concentration of the amine and the rate of addition of the amine solution (Y) were adjusted accordingly. The reaction medium was left stirring, while monitoring the reaction progress by infrared spectrometry (disappearance of the NCO band between 2250 and 2280 cm⁻¹).

Once the diisocyanate had completely reacted, the reaction mixture was added to water adjusted to pH 3 with hydrochloric acid, and the precipitate obtained was filtered off, washed several times with water, and finally dried. A white powder was obtained and was used without further purification after analysis by high-performance liquid chromatography (HPLC) coupled to mass spectrometry.

Compound (I) was obtained in the form of a mixture of compounds having the following formulae:

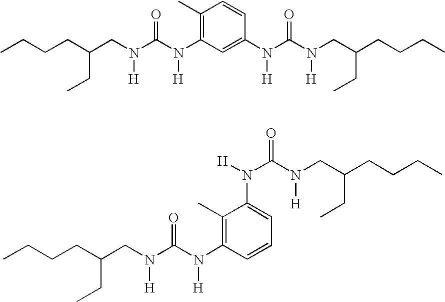

The molar ratio of the isomers was determined by ¹H nuclear magnetic resonance (NMR) spectrometry and/or by HPLC. The NMR spectra were consistent with the expected structures. The mixture of products obtained was in the form of a white powder.

2. Preparation of the Foundation Composition

The compounds of phase A1 were melted in a beaker, at a temperature of 100° C. with magnetic stirring for about one hour (phase A1).

The compounds of phase A2 were added to phase A1 with stirring using a Moritz blender, at a temperature of 80° C. (phases A1+A2).

The pigments of phase A3 were ground in cyclopentasiloxane in a three-roll mill, and phase A3 was then added to the mixture A1+A2.

The Nylon powder of phase A4 was added with continued stirring, by sprinkling it onto the mixture A1+A2+A3.

The water and the preserving agents were weighed out in a separate beaker. This beaker was placed on a hotplate with magnetic stirring in order to dissolve the preserving agents in the water. The mixture B was allowed to cool to room temperature and was then poured slowly into the mixture A1+A2+A3+A4 to form the emulsion.

The foundation obtained had a creamy texture, which, when applied to the skin, formed a smooth film. In addition, after taking up on a finger the product in the jar (or after manual shear), the surface of the product in the jar regained its initial shape (surface repair).

### EXAMPLE 2

A foundation having the composition below was prepared:

| | | Concentration |
|---|---|---|
| PHASE | Name | (mass %) |
| A1 | Compound of formula (I) in the form of a mixture of the | 2.50 |
| | compounds: | |
| | 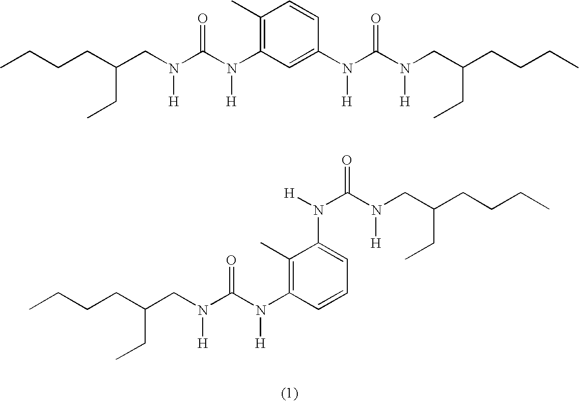 | |
| | Parleam oil | 31.10 |
| A2 | Cetyl dimethicone copolyol (2) | 1.80 |
| | Mono/diglycerides of isostearic acid esterified with succinic | 0.60 |
| | acid (3) | |
| A3 | Cyclopentasiloxane | 5.00 |
| | Iron oxide pigments | 2.04 |
| | Titanium dioxide pigments | 7.96 |
| A4 | Nylon 12 | 8.00 |
| B | Water | 40.00 |
| | Preserving agents | 1.00 |
| | TOTAL | 100.00 |

(1) Obtained according to the same method of preparation as Example 1.
(2) Sold under the name Abil EM 90 by the company Goldschmidt.
(3) Sold under the name Imwitor 780 K by the company Sasol.

The procedure was the same as that of Example 1.

The foundation obtained had a creamy texture, which, when applied to the skin, formed a smooth film. In addition, after taking up on a finger the product in the jar (or after manual shear), the surface of the product in the jar regained its initial shape (surface repair).

What is claimed is:

1. A cosmetic composition comprising, in a cosmetically-acceptable medium,
(i) at least one continuous liquid fatty phase, comprising at least one compound of formula (I): 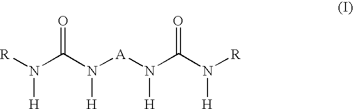
wherein:
A is a group of formula: 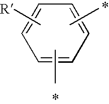
wherein R′ is chosen from linear and branched C₁ to C₄ alkyl radicals and the *s represent the points of attachment of the group A to each of the two nitrogen atoms of the rest of the compound of formula (I), and
R is chosen from saturated and unsaturated, non-cyclic, mono-branched alkyl radicals comprising from 6 to 15 carbon atoms, whose hydrocarbon-based chain may be optionally interrupted with 1 to 3 heteroatoms chosen from O, S, and N,
or a salt or isomer thereof, and
(ii) at least one non-silicone oil with a solubility parameter δₐ ranging from 0 to 5.00 (J/cm³)^{1/2}.

2. A composition according to claim 1, wherein A is a group of formula:

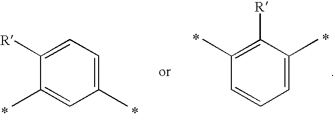

3. A composition according to claim 2, wherein A is a group of formula:

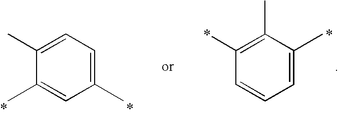

4. A composition according to claim 1, wherein R is of formula CₙH₂ₙ₊₁, wherein n is an integer ranging from 6 to 15.

5. A composition according to claim 4, wherein n ranges from 7 to 9.

6. A composition according to claim 5, wherein R is a group of formula: 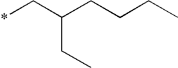
wherein * represents the point of attachment of R to the rest of the compound of formula (I).

7. A composition according to claim 1, wherein R is of formula Cₘ₋ₚH₂ₘ₊₁₋₂ₚXₚ, wherein
p is an integer ranging from 1 to 3,
m is an integer ranging from 6 to 15, and
X is chosen from S and 0.

8. A composition according to claim 7, wherein R is of formula C_{m′}H_{2m′}X—(C_{p′}H_{2p′}X′)—CₓH₂ₓ₊₁, wherein X and X′ are, independently of each other, chosen from O and S, r is an integer ranging from 0 to 1, and m′, p′, and x are integers ranging from 0 to 15, wherein the sum of m′, p′, and x ranges from 6 to 15.

9. A composition according to claim 8, wherein r is equal to 0.

10. A composition according to claim 8, wherein the chain CₓH₂x+₁ is branched.

11. A composition according to claim 8, wherein m′ is an integer ranging from 1 to 10.

12. A composition according to claim 8, wherein x is an integer ranging from 4 to 16.

13. A composition according to claim 7, wherein m is an integer ranging from 10 to 12.

14. A composition according to claim 8, wherein the sum of m′, p′, and x is an integer ranging from 10 to 12.

15. A composition according to claim 1, wherein R is a group of formula: 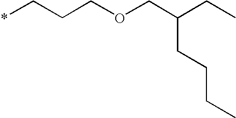
wherein * represents the point of attachment of R to the rest of the compound of formula (I).

16. A composition according to claim 1, wherein the composition comprises at least one compound chosen from: 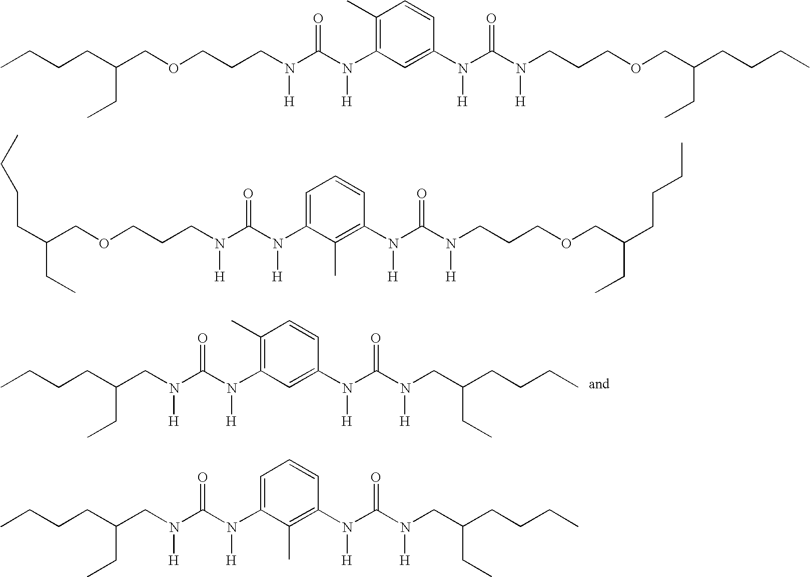
and salts and isomers thereof.

17. A composition according to claim 1, wherein the composition comprises 2% to 8% by weight of compound(s) of formula (I), relative to the total weight of the composition.

18. A composition according to claim 1, wherein said at least one compound of formula (I) is present in an amount less than or equal to 3% by weight, relative to the total weight of the liquid fatty phase.

19. A composition according to claim 1, wherein said non-silicone oil with a solubility parameter δₐ ranging from 0 to 5.00 (J/cm³)^{1/2} is chosen from squalane, parleam oil, polyisobutylene, jojoba oil, sesame oil, isopropyl myristate, butyl stearate, isononyl isononanoate, isopropyl palmitate, arachidyl propionate, stearyl heptanoate, isopropyl stearate, isostearyl neopentanoate, 2-ethylhexyl palmitate, cetyl 2-ethylhexanoate, isopropyl isostearate, C₁₂-C₁₅ alkyl benzoates, macadamia oil, octyidodecyl stearoylstearate, arara oil, PVP/hexadecene copolymer, oleyl erucate, octyidodecyl stearate, isostearyl palmitate, isocetyl stearate, didecene, hydrogenated polydecene, diisocetyl dodecanedioate, isostearyl isostearate, dicaprylyl ether, pentaerythrityl tetraisostearate, glyceryl triisostearate, octyidodecyl neopentanoate, isoeicosane, diisopropyl dimer dilinoleate, dioctyldodecyl dimer dilinoleate, octyldodecyl myristate, tridecyltetradecanoin, triisostearyl trilinoleate, isostearyl benzoate, isodecyl isononanoate, diisostearyl adipate, tridecyl isononanoate, triisopalmitine, 2-decyl hexyl isononanoate, 2-octyldodecyl benzoate, diisoarachidyl dodecanedioate, propylene glycol diisostearate, hydrogenated dimer dilinoleyl/dimethyl carbonate copolymer, octyldodecyl neodecanoate, octyldodecyl octanoate, isostearyl isononanoate, isofol-24 isostearate, octyldodecyl erucate, pentaerythrityl tetraoctyldodecanoate, pentaerythrityl tetradecyltetradecanoate, pentaeryth rityl tetrakis(2-hexyldecanoate), ditrimethylol-propane tetraisostearate, castor oil benzoate (ratio 1/1.5), octyldodecyl PPG-3 myristyl ether dimer dilinoleate and trimethylolpropane triisostearate, and mixtures thereof.

20. A composition according to claim 1, wherein said non-silicone oil with a solubility parameter δₐ ranging from 0 to 5.00 (J/cm³)^{1/2} is non-volatile.

21. A composition according to claim 1, wherein said non-silicone oil with a solubility parameter δₐ ranging from 0 to 5.00 (J/cm³)^{1/2} is aprotic.

22. A composition according to claim 1, wherein said non-silicone oil with a solubility parameter δₐ ranging from 0 to 5.00 (J/cm³)^{1/2} is chosen from alkanes, esters, ethers, and carbonates, and mixtures thereof.

23. A composition according to claim 22, wherein said non-silicone oil with a solubility parameter δₐ ranging from 0 to 5.00 (J/cm³)^{1/2} is chosen from alkanes comprising from 14 to 65 carbon atoms.

24. A composition according to claim 23, wherein said non-silicone oil with a solubility parameter δₐ ranging from 0 to 5.00 (J/cm³)^{1/2} is chosen from squalane, polyisobutylenes with a molecular weight ranging from 250 to 800 g/mol, and isoeicosane, and mixtures thereof.

25. A composition according to claim 24, wherein said non-silicone oil with a solubility parameter δₐ ranging from 0 to 5.00 (J/cm³)^{1/2} is parleam oil.

26. A composition according to claim 22, wherein said non-silicone oil with a solubility parameter δₐ ranging from 0 to 5.00 (J/cm³)^{1/2} is chosen from esters, ethers, and carbonates, and mixtures thereof, and has a molecular mass ranging from 250 to 3000 g/mol.

27. A composition according to claim 1, wherein said non-silicone oil with a solubility parameter δₐ ranging from 0 to 5.00 (J/cm³)^{1/2} is present in an amount ranging from 20% to 100% by weight, relative to the total weight of the continuous liquid fatty phase.

28. A composition according to claim 1, further comprising at least one additional oil which is different from said non-silicone oil with a solubility parameter δₐ ranging from 0 to 5.00 (J/cm³)^{1/2} and which is chosen from volatile oils, non-volatile oils, and mixtures thereof.

29. A composition according to claim 28, wherein said additional oil is present in an amount ranging from 0.1% to 15% by weight, relative to the total weight of the composition.

30. A composition according to claim 1, further comprising an aqueous phase.

31. A composition according to claim 1, wherein water and oils are present in amounts such that the weight ratio of water to oils is greater than or equal to 1.

32. A composition according to claim 1, further comprising a pulverulent phase comprising pigments, fillers, and nacres, and/or mixtures thereof.

33. A composition according to claim 1, further comprising at least one wax.

34. A composition according to claim 1, further comprising at least one mineral thickener.

35. A composition according to claim 1, wherein said composition is in anhydrous form, or in the form of water-in-oil or multiple emulsions having a continuous liquid fatty phase, and is in the form of gels, creams, or suspensions, in compact or hot-cast form, or in the form of sticks.

36. A process for making up or caring for keratin materials, comprising applying to said keratin materials at least one composition comprising, in a cosmetically-acceptable medium,
(i) at least one continuous liquid fatty phase, comprising at least one compound of formula (I): 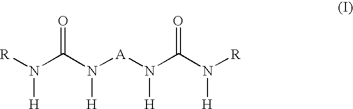
wherein:
A is a group of formula: 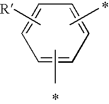
wherein R′ is chosen from linear and branched C₁ to C₄ alkyl radicals and the *s represent the points of attachment of the group A to each of the two nitrogen atoms of the rest of the compound of formula (I), and
R is chosen from saturated and unsaturated, non-cyclic, mono-branched alkyl radicals comprising from 6 to 15 carbon atoms, whose hydrocarbon-based chain may be optionally interrupted with 1 to 3 heteroatoms chosen from O, S, and N,
or a salt or isomer thereof, and
(ii) at least one non-silicone oil with a solubility parameter δₐ ranging from 0 to 5.00 (J/cm³)^{1/2}.
